(19) United States
(12) Patent Application Publication
(10) Pub. No.: US 2007/0059746 A1
(43) Pub. Date: Mar. 15, 2007

(54) Substance carrier using hollow nanoparticle of hepatitis B virus protein and liposome, and method of introducing substance into cell

(75) Inventors: Shun-ichi Kuroda, Suita-shi (JP); Atsuko Ueda, Suita-shi (JP); Mana Nagita, Okayama-shi (JP)

Correspondence Address: KNOBBE MARTENS OLSON & BEAR LLP, 2040 MAIN STREET, FOURTEENTH FLOOR, IRVINE, CA, 92614, US

(73) Assignees: Japan Science and Technology Agency, Kawaguchi-shi (JP); Osaka University, Suita-shi (JP); Beacle Inc., Okayama-shi (JP)

(21) Appl. No.: 11/515,319

(22) Filed: Sep. 1, 2006

(30) Foreign Application Priority Data
Sep. 14, 2005 (JP) 2005-267573

(51) Int. Cl. C12Q 1/70; C12Q 1/68

(52) U.S. Cl. 435/6; 435/5

(57) ABSTRACT

The present invention relates to a method of producing a composite particle of a nanoparticle and a liposome in which a substance to be introduced has been encapsulated, characterized in that a hollow nanoparticle containing a hepatitis B virus protein or a modification thereof is fused to the liposome in which the substance to be introduced has been encapsulated.

### TECHNICAL FIELD

The present invention relates to a method of encapsulating a substance into a hollow nanoparticle which is a carrier capable of introducing the substance into a certain cell or tissue, and a composite particle which has encapsulated the substance to be introduced.

### BACKGROUND ART

In recent years, a method referred to as a drug delivery system (DDS) to reduce side effects of drugs has been noticed. The DDS is the method in which an active component such as a drug is specifically carried by a carrier to an objective cell or tissue at a diseased site and allows to act at the objective site. The present inventors have found that a particle (HBsAg particle) composed of a hepatitis B virus surface antigen protein, into which a biorecognition molecule has been introduced is effective as a DDS carrier for specifically and safely carrying and delivering the substance to be introduced to the objective portion (WO01/64930, WO03/082330, WO03/082344). To encapsulate the substance such as drugs, genes and proteins to be introduced in the particle, conventionally an electroporation method has been used. When the electroporation is performed, an apparatus specific for the electroporation and technical knowledge for manipulation of the apparatus are required. Thus, the method of encapsulating the substance into a hollow nanoparticle by the electroporation has had a certain restriction.

As shown in WO97/17844, it has been known that a Sendai virus particle containing about 70% lipid as a major component easily forms a composite particle with liposome which is the lipid to encapsulate the substance into the viral particle, but no interaction between the HBsAg protein and the liposome has been known.

### DISCLOSURE OF THE INVENTION

It is an object of the present invention to provide a composite particle for specifically, efficiently and safely carrying and introducing a substance into a target cell or tissue, and a method of producing the same.

As a result of an extensive study in the light of the above problem, the present inventor has found that even a hollow nanoparticle such as an HBsAg particle whose major component is the protein can be fused to the liposome by mixing the liposome to form the composite particle and the hollow nanoparticle can encapsulate the substance therein.

The present invention relates to the following composite particles and methods of producing the same.

[1] A method of producing a composite particle of a nanoparticle and a liposome encapsulating a substance to be introduced, characterized in that a hollow nanoparticle comprising a hepatitis B virus protein or a modification thereof is fused to the liposome in which the substance has been encapsulated.

[2] The method according to [1] above wherein a particle diameter of the hollow nanoparticle is about 80 to about 130 nm.

[3] The method according to [1] above wherein a particle diameter of the composite particle is about 130 to about 500 nm.

[4] The method according to [1] above wherein a particle diameter of the composite particle is about 150 to about 400 nm.

[5] The method according to [1] above wherein the hollow nanoparticle comprising the hepatitis B virus protein or the modification thereof is composed of about 70 to about 90 parts by weight of the hepatitis B virus protein or the modification thereof, about 5 to about 15 parts by weight of lipid and about 5 to about 15 parts by weight of sugar chain.

[6] The method according to any of [1] to [5] above wherein the hollow nanoparticle has been previously lyophilized or spray-dried.

[7] A composite particle comprising a nanoparticle portion composed of a hepatitis B virus protein or a modification thereof, lipid and sugar chain, and an exogenous substance encapsulated in the nanoparticle portion.

[8] The composite particle according to [7] above wherein a particle diameter of the composite particle is about 150 to about 400 nm.

[9] The composite particle according to [7] or [8] above wherein the nanoparticle portion comprises about 70 to about 90 parts by weight of the hepatitis B virus protein or the modification thereof, about 6 to about 75 parts by weight of the lipid and 5 to 15 parts by weight of the sugar chain.

[10] The composite particle according to [9] above wherein said lipid comprises about 5 to about 15 parts by weight of the lipid which is the component of a membrane of an eukaryotic cell and about 1 to about 60 parts by weight of the lipid which is the component of a liposome.

[11] The composite particle according to [9] above wherein the nanoparticle portion comprises about 70 to about 90 parts by weight of the hepatitis B virus protein or the modification thereof, about 5 to about 15 parts by weight of lipid which is the component of a membrane of an eukaryotic cell, about 2 to about 30 parts by weight of lipid which is the component of a liposome and about 5 to about 15 parts by weight of the sugar chain.

[12] A composite particle of a nanoparticle and a liposome encapsulating a substance to be introduced, obtainable by the method according to any of [11 to [6] above.

[13] A method of introducing a substance into a target cell, including allowing the composite particle according to any of [7] to [11] above or the composite particle according to [12] above to act upon the target cell.

According to the present invention, the substance can be easily encapsulated in the hollow nanoparticle such as an HBsAg particle which has a high protein content and is rigid. The substance to be introduced may be the substance having a size of about 100 nm to about 500 nm which is comparative to or much larger than the hollow nanoparticle before encapsulation.

In the composite particle of the present invention in which the substance has been encapsulated using the liposome, an introduction efficiency of the substance is enhanced compared with the hollow particle in which the substance has been encapsulated by electroporation.

Furthermore, by encapsulating DNA or RNA such as plasmids in the liposome followed by being fused with the hollow nanoparticle, it is possible to make it the composite particle which is smaller than original size of DNA or RNA.

By the use of the composite particle of the present invention, it becomes possible to specifically introduce the substance into the particular cell and tissue in vivo or in vitro.

### BRIEF DESCRIPTION OF THE DRAWINGS

FIG. 1A is a graph showing the separation by ultracentrifugation of HBsAg fusion particles resulted from the fusion of hollow nanoparticles and liposomes encapsulating a substance in Example of the present invention;

FIG. 1B Electron-micrographs of liposome (left), BNC (middle), and BNC fused with liposome containing 100-nm polystyrene beads (right) were observed using TEM, following negative staining. Scale bar, 100 nm;

FIG. 2 is a graph showing the result of quantifying amounts of FITC-labeled 100-nm polystyrene beads in respective cells when HBsAg fusion particles resulted from encapsulating the FITC-labeled 100-nm polystyrene beads into hollow nanoparticles via the liposome were contacted with human hepatic cancer derived cell HepG2 and human large intestine cancer derived cell WiDr as a control, respectively in Example of the present invention. RFU represents a relative fluorescent unit;

FIG. 3 shows confocal laser fluorescent micrographs of HepG2 and WiDr when the HBsAg fusion particles resulted from encapsulating the FITC-labeled 100-nm polystyrene beads into hollow nanoparticles via the liposome were contacted with HepG2 and WiDr;

FIG. 4 shows confocal laser fluorescent micrographs showing that the HBsAg fusion particles encapsulating the FITC-labeled 100-nm polystyrene beads can introduce the FITC-labeled 100-nm polystyrene beads highly specifically into human hepatic cancer derived cell NuE. A tumor portion (with fluorescence) from a tumor-bearing mouse in which NuE was transplanted and normal liver (with no fluorescence) from a mouse are shown;

FIG. 5 shows the results of quantifying the amounts of the FITC-labeled 100-nm polystyrene beads in human squamous cell carcinoma derived cell line A431. A ZZ-HBsAg fusion particle resulted from encapsulating a ZZ tag which was a biorecognition molecule having a binding capacity with an antibody into the hollow nanoparticle via the liposome containing the FITC-labeled 100-nm polystyrene beads was bound to a monoclonal antibody (anti-hEGFR antibody) against human epidermal growth factor receptor (hEGFR) to yield an anti-hEGFR antibody-presenting ZZ-HBsAg fusion particle. This anti-hEGFR antibody-presenting ZZ-HBsAg fusion particle was contacted with the human squamous cell carcinoma derived cell line A431. As the control, the result obtained from an antibody non-presenting ZZ-HBsAg fusion particle was shown together;

FIG. 6 shows confocal laser fluorescent micrographs of A431 cells. The ZZ-HBsAg fusion particle resulted from encapsulating the FITC-labeled 100-nm polystyrene beads into a ZZ-HBsAg particle via the liposome was bound to the anti-hEGFR antibody to yield the anti-hEGFR antibody presenting ZZ-HBsAg fusion particle. This anti-hEGFR antibody presenting ZZ-HBsAg fusion particle was contacted with A431. As the control, the result obtained from the antibody non-presenting ZZ-HBsAg fusion particle was shown together;

FIG. 7 shows confocal laser fluorescent micrographs of A431 cells and MCF-7 cells. The ZZ-HBsAg fusion particle resulted from encapsulating the FITC-labeled 100-nm polystyrene beads into a ZZ-HBsAg particle via the liposome was bound to the anti-hEGFR antibody to yield the anti-hEGFR antibody presenting ZZ-HBsAg fusion particle. This anti-hEGFR antibody presenting ZZ-HBsAg fusion particle was contacted with the A431 cell or human breast cancer derived cell MCF-7 cell; and

FIG. 8 shows confocal laser fluorescent micrographs of HepG2 and WiDr when an HBsAg fusion particle resulted from encapsulating a green fluorescent protein (GFP) expressing gene into the hollow nanoparticle via the liposome was contacted with the human hepatic cancer derived cell line HepG2 and the human large intestine cancer cell line WiDr as the control.

FIG. 9 Ex vivo delivery of rhodamine-labeled 100-nm polystyrene beads (Rho-beads) with BNC fused liposome. Rho-beads were encapsulated into BNC fused liposome. These BNCs were applied to HepG2 cells and WiDr cells and incubated for 6 h. A) Cells were observed under a confocal microscope. Scale bar, 50 μm. B) RFU of the cells was measured with a microplate reader.

FIG. 10 In vivo delivery of Rho-beads with BNC fused liposome. Rho-beads were encapsulated into BNC fused liposome. These BNCs were injected (i.v.) in the mouse xenograft model (the nude mice bearing NuE cell-derived tumor and A431 cell-derived tumor. After 16 h, FITC-labeled tomato lectin was injected (i.v.) and scarify. Fluorescence was observed in sections from NuE-derived tumor. Scale bar, 100 μm.

FIG. 11 Ex vivo gene delivery with BNC fused liposome. GFP plasmid was encapsulated into BNC fused liposome. These BNCs were applied to HepG2 and WiDr cells. After 48 h, the expression of GFP was observed under a confocal microscope (A) and RFU was calculated by Imaging J software (B). Scale bar, 100 μm.

FIG. 12 In vivo gene delivery with BNC fused liposome. GFP plasmid was encapsulated into BNC fused liposome. These BNCs (50 μg) were injected (i.v.) into the mouse xenograft model (the nude mice bearing both NuE and A431 cell-derived tumors). After 7 days, fluorescence was observed in sections from tumors. Scale bar, 100 μm.

FIG. 13 Ex vivo Large plasmid delivery with BNC fused liposome. 35-kbp GFP plasmid was encapsulated into BNC fused liposome. These BNCs were applied to HepG2 and A431 cells. After 48 h, the expression of GFP was observed under a confocal microscope. Scale bar, 100 μm.

FIG. 14 The efficiencies of incorporation methods. 100-nm polystyrene beads (A), and pcDNA/CMV-GFP (˜35 kbp) (B) were transfected in HepG2 cells by electroporation (EP, white squire) and fusion of BNC with liposome (Fusion, black squire).

### BEST MODES FOR CARRYING OUT THE INVENTION

Herein, as the hollow nanoparticle comprising the hepatitis B virus protein or the modification thereof, in which the substance to be introduced is encapsulated, the HBsAg protein particle and the like are exemplified. The particle may be formed by combining the HBsAg protein with a hepatitis B virus basal core antigen protein.

Herein, sizes of the composite particle, the hollow nanoparticle and the substance to be introduced (nucleic acids, proteins and drugs) may be measured by an electron microscopy or optically measured by a zeta sizer nano-ZS (Malvern Instruments).

The hollow nanoparticle for encapsulating the substance to be introduced in the present invention may contain the hepatitis B virus protein as the major component, and the protein may have a sugar chain. A lipid component may also be contained in the hollow nanoparticle.

In one preferable embodiment, the hollow nanoparticle comprises about 70 to about 90 parts by weight of the hepatitis B virus protein or the modification thereof, about 5 to about 15 parts by weight of the lipid which is the component of an endoplasmic reticulum membrane of a eukaryotic cell and 5 to 15 parts by weight of the sugar chain. The hollow nanoparticle used in Examples in the present application comprises about 80 parts by weight of the hepatitis B virus protein, about 10 parts by weight of the sugar chain and about 10 parts by weight of the lipid (J Biotechnol. November 1992;26(2-3):155-62. Characterization of two differently glycosylated molecular species of yeast-derived hepatitis B vaccine carrying the pre-S2 region. Kobayashi M, Asano T, Ohfune K, Kato K.). Sendai virus publicly known to be fused with the liposome has the lipid as the major component and has the structure in which a small amount of the protein is floated on the liposome (Methods Enzymol. 1993;221:18-41. Okada Y. Sendai virus-induced cell fusion.). It is believed that Sendai virus can be fused with liposome by a membrane-fusing activity of F protein. The inventors surprisingly found that a hepatitis B virus protein had a membrane-fusing activity capable of fusion between the hollow nanoparticle of the present invention and liposome.

In the preferable embodiments of the present invention, the nanoparticle portion encapsulating a complex after the fusion with the liposome (structure portion other than the encapsulated substance such as a complex) can comprise about 70 to about 90 parts by weight of the hepatitis B virus protein or the modification thereof, about 6 to about 75 parts by weight of the lipid (comprising 5 to 15 parts by weight of lipid which is the component of a membrane of an eukaryotic cell and about 1 to about 60 parts by weight of lipid which is the component of a liposome) and about 5 to about 20 parts by weight of the sugar chain.

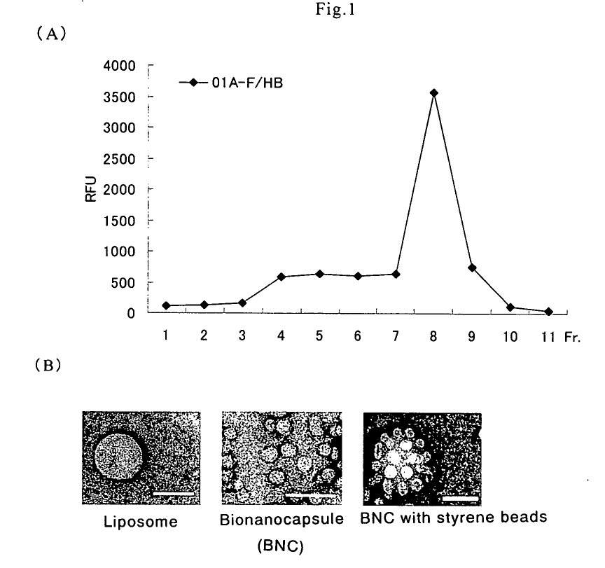

The composite particle of the present invention may have the structure in which at least one liposome and lipid portion of at least one hollow nanoparticle are partially or completely fused wherein the hepatitis B virus protein or the modification thereof is penetrated into lipid membrane of the fused particle. One example of fused particle is shown in FIG. 1B.

In the preferable embodiments of the present invention, the nanoparticle portion encapsulating the substance in the composite particle after being fused with the liposome (structural portion other than the encapsulated substance) can comprise about 70 to about 85 parts by weight of the hepatitis B virus protein or the modification thereof, about 5 to about 15 parts by weight of lipid which is the component of a membrane of a eukaryotic cell, 2 to 30 parts by weight of lipid which is the component of a liposome and 5 to 15 parts by weight of the sugar chain. Liposome usually comprises phospholipids and other components such as cholesterol, wherein said other components are not more than about 20% by weight based on total weight of liposome. The lipid of the hollow nanoparticle is derived from endoplasmic reticulum membrane of eukaryotic cells such as mammalian cells (CHO cell, HEK293 cell, COS cell, etc), yeast and insect cells (Sf9 cell, Sf21 cell, HighFive cell). The lipid of the hollow nanoparticle is mainly composed of phospholipids such as phosphatidylcholine (PC), phosphatidylethanolamine (PE), phosphatidylserine (PS), etc. Other components of the hollow nanoparticle may include cholesterol.

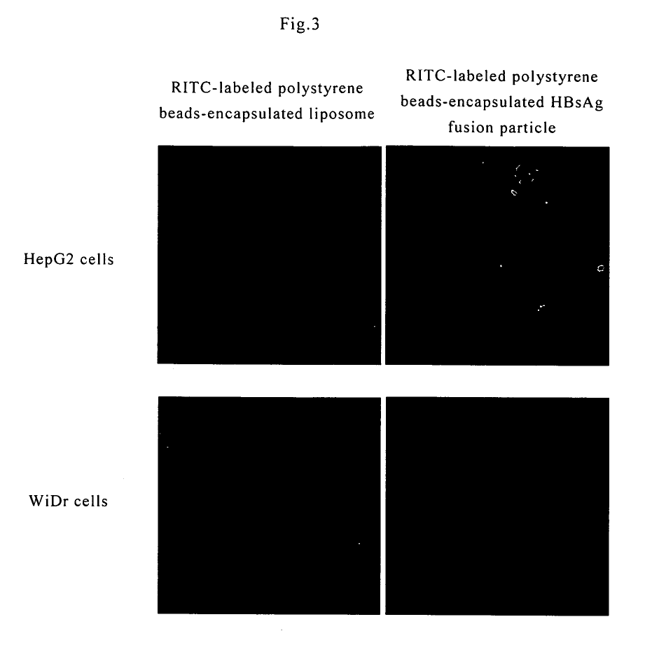

As shown in FIG. 3, in the composite particle of the present invention, an introduction efficiency of the substance is much more excellent compared with the case of the electroporation.

Herein, the “hollow nanoparticle” means the particle before the substance is encapsulated, and the “nanoparticle” means the particle after the substance has been encapsulated. The “composite particle” indicates the nanoparticle in which the substance has been encapsulated by fusing the hallow nanoparticle to the liposome encapsulating the substance.

An S protein (226 amino acid residues) which is included in HBsAg and is the component of an S particle has a particle forming ability. An M protein (constitutive protein of M particle) is obtained by adding Pre-S2 composed of 55 amino acid residues to the S particle. An L protein (constitutive protein of L particle) is obtained by adding Pre-S1 composed of 108 amino acid residues (subtype y) or 119 amino acid residues (subtype d) to the M protein. The L protein and the M protein have the particle forming ability similarly to the S protein. Therefore, two regions of Pre-S1 and Pre-S2 may be optionally substituted, added, deleted or inserted. For example, the hollow particle which lacks a hepatic cell recognition ability can be obtained by using a modified protein obtained by deleting a hepatic cell recognition site contained at positions 3 to 77 in the Pre-S1 region (subtype y). Since the hepatic cell recognition site through albumin is contained in the Pre-S2 region, this albumin recognition site can also be deleted. Meanwhile, since the S region (226 amino acid residues) bears the particle forming ability, it is necessary to modify the S region so that the particle forming ability is not impaired. For example, since the particle forming ability is retained when the positions 107 to 148 in the S region is deleted (J. Virol. 2002 76 (19), 10060-10063), this region may be substituted, added, deleted or inserted. When hydrophobic 154 to 226 residues at the C terminus are substituted, added, deleted or inserted, the particle forming ability can also be retained. Meanwhile, 8 to 26 residues (TM1) and 80 to 98 residues (TM2) are transmembrane helix (transmembrane sequence). Thus, it is desirable that this region is not mutated or is deleted, added or substituted by leaving the hydrophobic residues so that the transmembrane property is retained.

In one preferable embodiment, the modification of the hepatitis B virus protein widely includes various modifications as long as they have the ability to form the hollow nanoparticle. Taking HBsAg as an example, any numbers of substitutions, deletions, additions and insertions are included for the Pre-S1 and Pre-S2 regions. For the S region, one or more, e.g., 1 to 120, preferably 1 to 50, more preferably 1 to 20, still more preferably 1 to 10 and particularly 1 to 5 amino acid residues may be substituted, added, deleted or inserted. Methods of introducing mutations such as substitution, addition, deletion and insertion include gene engineering techniques such as site specific mutagenesis (Methods in Enzymology, 154, 350, 367-382 (1987); ibid., 100, 468 (1983);Nucleic Acids Res., 12, 9441 (1984)) and chemical synthesis means such as phosphate triester method and phosphate amidite method (e.g., using a DNA synthesizer) (J. Am. Chem. Soc., 89, 4801 (1967); ibid., 91, 3350 (1969);Science, 150, 178 (1968); Tetrahedron Lett., 22, 1859 (1981)). Selection of codons can be determined in consideration of codon usage in a host.

In the case of the hollow bionanoparticle composed of hepatic cell-recognizable proteins such as hepatitis B virus protein or modification thereof such as L protein and M protein, it is not necessary to introduce the cell recognition site. On the other hand, in the case of the hollow bionanoparticle composed of the modified protein obtained by deleting the hepatic cell recognition site contained at 3 to 77 amino acid residues in the PreS1 region (in the case of subtype y) or the protein obtained by deleting both PreS1 and PreS2 regions, the bionanoparticle can not directly recognize the cell, and thus, the cell recognition site is introduced to have it recognize the optional cell other than the hepatic cell, for example, leading to being capable of introducing nucleic acids into various target cells. For such a cell recognition site which recognizes the particular cell, for example, cell function regulatory molecules composed of polypeptides such as growth factors and cytokines, cell surface antigens, histocompatibility antigens, polypeptide molecules such as receptors which discriminate the cell and the tissue, polypeptide molecules derived from viruses and microorganisms, antibodies and sugar chains can be preferably used. Specifically, the antibodies against EGF receptor and IL-2 receptor which appear specifically on cancer cells, or EGF, or receptors presented by HBV are included. Alternatively, proteins (e.g., ZZ tag) capable of binding an antibody Fc domain, or strepto tag which exhibits a biotin-like activity for presentong a biorecognition molecule labeled with biotin which is recognized via streptoavidin can also be used.

The above ZZ tag is defined as the amino acid sequence having the ability to bind to the Fc region of immunoglobulin and composed of the following twice repeated sequence (sequence of ZZ tag:

VDNKFNKEQQNAFYEILHLPNLNEEQRNAFIQSLKDDPSQSANLLAEAKK
LNDAQAPKVDNKFNKEQQNAFYEILHLPNLNEEQRNAFIQSLKDDPSQSA
NLLAEAKKLNDAQAPK)

When the cell recognition site is the polypeptide, the hollow bionanoparticle which recognizes the optional target cell can be obtained by ligating DNA encoding the hepatitis B virus protein or the modification thereof to DNA encoding the cell recognition site in frame through DNA encoding a spacer peptide as needed, incorporating this into a vector and expressing it in an eukaryotic cell.

When the cell recognition site is the antibody, the objective hollow bionanoparticle can be obtained by ligating DNA encoding the hepatitis B virus protein or the modification thereof to DNA encoding the ZZ tag in frame through DNA encoding the spacer peptide as needed, incorporating this into the vector, expressing it in the eukaryotic cell, and mixing the resulting hollow bionanoparticle with the antibody capable of recognizing the target cell.

When the cell recognition site is the sugar chain, the objective hollow bionanoparticle can be obtained by attaching biotin to a hollow nanoparticle, followed by treating the biotin-modified hollow bionanoparticle having no cell recognition site with streptavidin and one or more biotin-labeled sugar chains resulting in the hollow bionanoparticle presenting the sugar chain.

The modifications of the HBsAg protein may be the modifications obtained by modifying antigenicity (modifications obtained by deleting/substituting the site such as epitope involved in the antigenicity), stability of a particle structure and cell selectivity.

The size of the hollow nanoparticle is about 50 to about 500 nm and preferably about 80 to about 130 nm. It is desirable that the hollow nanoparticle is large above some extent when the substance to be introduced is large. To enlarge the hollow nanoparticle, the longer one of the hepatitis B virus protein or the modification thereof which has a Pre-S region longer than 163 amino acids (Pre-S1+Pre-S2) in the case of subtype y and which protein composes the particle could be used. For example, when the hepatic cell is targeted, the L particle or the hollow bionanoparticle having the size equivalent thereto is preferably used. The hollow nanoparticle which targets the cell other than the hepatic cell can be obtained by introducing the cell recognition site into the protein obtained by deleting the hepatic cell recognition site contained in S protein/M protein or at 3 to 77 amino acid residues in the case of subtype y in the PreS1 region of L-protein.

The hollow nanoparticle include those obtained by expressing the HBsAg protein in the eukaryotic cell. The method of producing the hollow nanoparticle is described in WO01/64930, WO03/082330 and WO03/082344, and the method of preparing HBsAg is described in Vaccine. Apr. 30, 2001;19(23-24):3154-63. Physicochemical and immunological characterization of hepatitis B virus envelope particles exclusively consisting of the entire L (pre-S1+pre-S2+S) protein. Yamada T, Iwabuki H, Kanno T, Tanaka H, Kawai T, Fukuda H, Kondo A, Seno M, Tanizawa K, Kuroda S.

When the HBsAg protein is expressed in the eukaryotic cell, the protein is expressed and accumulated as a membrane protein on an endoplasmic reticulum membrane and released as the nanoparticle, which is thus preferable. Animal cells such as mammalian cells and yeast cells can be applied as the eukaryotic cells. Such a particle is highly safe for human bodies because HBV genome is not contained at all. The cell selectivity of the nanoparticle of the present invention for the hepatic cell or the other cell can be enhanced by introducing the cell recognition site into at least a portion of the protein which composes the particle as needed.

In the method of producing the particle of the present invention, it is particularly preferable that the hollow nanoparticle whose component is the HBsAg protein is lyophilized followed by being fused with the liposome to make the composite particle. Spray drying can be used in place of the lyophilization. A fusion efficiency is remarkably enhanced by using the lyophilized or spray dried hollow nanoparticles.

In one embodiment of the present invention, the nanoparticle whose component is the HBsAg protein is once lyophilized or spray dried, and then mixed with the liposome to make the composite particle. Thus the substance encapsulated in the liposome can be incorporated inside the hollow nanoparticle. In the conventional method, the substance to be introduced has been incorporated into the hollow nanoparticle by electroporation, but in the method of the present invention, the substance to be introduced can be incorporated more easily into the hollow nanoparticle, and an introduction efficiency into the cell is also enhanced.

It is also possible to perform freezing, rapid thawing and heat treatment in place of the lyophilization or the spry drying.

For a ratio of the hollow nanoparticle lyophilized as needed and the liposome, for example, about 0.1 to about 10 mg, preferably about 0.5 to about 2 mg of the liposome is used per 1 mg of the hollow nanoparticle (lyophilized). Their mixture can be easily performed by mixing at about 37° C. for about 10 minutes. When the amount of the liposome to be used is too large relative to the hollow nanoparticle, the introduction efficiency of the substance to be introduced is lowered. Meanwhile when the amount of the liposome is too small, the efficiency to encapsulate the substance into the hollow nanoparticle is lowered.

In the preferable embodiments of the present invention, the particle diameter of the hollow nanoparticle before forming the composite particle together with the liposome is about 80 to about 130 nm, and the particle diameter of the composite nanoparticle (composite particle) after forming the composite particle together with the liposome and incorporating the substance inside the particle is about 130 to about 500 nm, for example, about 150 to about 400 nm, and more preferably about 200 to about 400 nm.

The liposome may be either a multilayer liposome or a single membrane liposome. The size of the liposome is about 50 to about 300 nm (e.g., about 100 to about 300 nm), preferably about 80 to about 250 nm, more preferably about 100 to about 200 nm and particularly preferably about 100 to about 150 nm. It is preferable that the size of the liposome is about 0.5 to about 2 times as large as the size of the hollow nanoparticle.

The formation of the smooth composite particle is prevented when the liposome is too small or too large relative to the nanoparticle.

The liposome can be produced by a sonication method, a reverse phase evaporation method, a freezing thawing method, a spray drying method and the like.

The component of the liposome includes phospholipid, cholesterols and fatty acids. Specific examples thereof include natural phospholipids such as phosphatidyl choline, phosphatidyl serine, phosphatidyl glycerol, phosphatidyl inositol, phosphatidyl ethanolamine, phosphatidic acid, cardiolipin, sphingomyelin, egg yolk lecithin, soy bean lecithin and lysolecithin, or those obtained by hydrogenating them by standard methods, and synthetic phospholipids such as distearoylphosphatidyl choline, dipalmitoylphosphatidyl choline, dipalmitoylphosphatidyl ethanolamine, dipalmitoylphosphatidyl serine, eleostearoylphosphatidyl choline, eleostearoylphosphatidyl ethanolamine and eleostearoylphosphatidyl serine. It is preferable to use phospholipid by combining lipids having various saturation degrees. Additionally, cholesterols include cholesterol and phytosterol, and the fatty acids include oleic acid, palmitoleic acid, linoleic acid, or fatty acid mixtures containing these unsaturated fatty acids. The liposome containing the unsaturated fatty acid with small side chain is effective for producing the small liposome due to a curvature.

Specifically describing an example of the method of producing the liposome, the liposome encapsulating the substance to be introduced can be obtained by, for example, dissolving the phospholipid or cholesterol in an appropriate solvent, placing this in an appropriate container and distilling off the solvent under reduced pressure to form a phospholipid membrane inside the container, and adding an aqueous solution, preferably buffer containing the substance to be introduced thereto and stirring it. The composite particle of the liposome and the nanoparticle can be obtained by mixing the liposome directly or after once being lyophilized with the lyophilized nanoparticle of the present invention.

The composite particle of the present invention is useful as one for specifically delivering the substance to the particular cell. For example, if the composite particle is administered in the body by intravenous injection, the particle is circulated in the body, led to the target cell by the substance selective/specific for the hepatic cell or the other cell, which is presented on the particle surface, and the substance is introduced into the target cell.

The composite particle of the present invention can be preferably used as a cell introduction reagent by mixing with the target cell in vitro.

The substance introduced into the cell is not particularly limited, and examples thereof can include various medicaments which elicit a physiological action when introduced into the cell; physiologically active proteins such as such as hormones, lymphokines and enzymes; antigenic proteins which act as a vaccine; polynucleotides such as genes and plasmids which are expressed in the cells; polynucleotides which elicit or induce the expression and are involved in the particular gene expression, and various genes and antisense DNA/RNA introduced for gene therapy. The introduced “genes” include not only DNA but also RNA. As the introduced substance, physiologically active macromolecular substances such as proteins and genes can be preferably exemplified, but the preferable result can be obtained even when various medicaments with low molecular weight are applied. The gene and the proteins may also be natural or synthetic, or modified genes or proteins.

### EXAMPLES

Modes for carrying out the present invention will be described in more detail with reference to the following Examples along the accompanying drawings. Of course, the present invention is not limited to the following Examples, and it goes without saying that various aspects are possible in detail.

In the following Examples, HBsAg indicates a Hepatitis B virus surface antigen. HBsAg is expressed and accumulated as the membrane protein on the endoplasmic reticulum membrane when expressed in the eukaryotic cell. Subsequently, intermolecular aggregation occurs, and is released as an HBsAg particle at a lumen side by a budding mode with incorporating the endoplasmic reticulum membrane.

The HBsAg particles were obtained by expressing the HBsAg particles in the eukaryotic cells such as yeast cells, insect cells and mammalian cells and then purifying them (Patent Documents 1 to 3).

Experimental Procedures

Materials

BNC were produced from yeast as following the method of a previous paper (Kuroda S) and purified by using AKTA (Amershambiosciences co., Japan). Liposomes (Coatsome EL-01-A, Coatsome EL-01-D) were purchased from NOF corporation (Tokyo, Japan). Fluospheres carboxylate-modified microspheres (100-nm polystyrene beads) were purchased from Molecular probes Inc. (Eugene, Oreg.). Plasmid pEGFP-C1 was purchased form Clonetech laboratories Inc. (Takara bio Inc., Japan), and plasmid pcDNA6.2/C-EmGFP and pAD/CMV-GFP were purchased form Invitrogen co. (Carlsbad, Calif.).

Fusion of BNC and Liposome

To encapsulate 100-nm polystyrene beads into liposome, the freeze-dried liposomes (Coatsome-El-01-A) were solved with the aqueous solution of 100-nm polystyrene beads (0.2% w/v) and then gentle shaking at room temperature. For the encapsulation of DNA into liposome, the freeze-dried liposomes (Coatsome-EL-01-A) were solved with the aqueous solution of DNA (250 μg/mL) at room temperature for 15 min. Liposomes containing payloads (100-nm polystyrene beads or genes) were added to freeze-dried BNC at room temperature for 15 min.

Transmission Electron Microscopy (TEM)

To visually examine the BNC fused liposome, negative staining TEM was employed. Specimens were dropped on TEM grid treated hydrophilicity, stained with 2% phosphotungstic acid, and observed by TEM (JEOL, Japan).

Particle Size

The particle size was measured at 25° C., using a Zetasizer Nano-ZS (Malvern Instruments Ltd., U.K.). This measurement is based on a dynamic light scattering method; z-average particle size is estimated using the Einstein-Stokes equation.

Cells

HepG2 (human hepatocellular carcinoma) cells and A431 (human epidermoid carcinoma) cells were maintained in Dulbecco's modified Eagle medium supplemented with 10% fetal bovine serum (FBS). WiDr (human colon adenocarcinoma) and NuE (human hepatocellular carcinoma, obtained from T. Tadakuma, National Defense Medical College) cells were maintained in RPMI 1640 medium supplemented with 10% FBS. These cells were incubated at 37° C. in 5% CO₂.

Mouse Xenograft Model

Five-week-old male BALB/c nude (nu/nu) mice were purchased from CLEA Japan, Inc. (Osaka, Japan). Animals were treated according to the guideline of the Ministry of Education, Culture, Sports, Science and Technology, Japan. About 5×10⁶ carcinoma cells (NuE and A431) were subcutaneously injected into the backs of the mice. After about 2 weeks, mice were injected with BNC containing 100-nm polystyrene beads or GFP plasmid intravenously.

Histological Analyses

The mice were anesthetized with pentobarbital (Dainippon sumitomo pharma Co., Japan) and tumors, livers, and kidneys were isolated. These tissues were fixed in 4% (wt/vol) para-formaldehyde and embedded in the synthetic resin with Technovit 8100 (Kluzer, Germany). The blocks were sectioned into a width of 5 μm and then observed under a LSM5 PASCAL laser scanning confocal microscope (Carl ziess, Germany).

### Example 1

### Encapsulation of Substance into HBsAg Particle Via Liposome

An FITC-labeled 100-nm polystyrene beads-encapsulated liposome solution was prepared by adding 1 mL of an FITC-labeled 100-nm polystyrene bead solution prepared at 10 mg/mL from FITC-labeled 100-nm polystyrene beads (Fluospheres^{R} [diameter: 100 nm] supplied from Molecular Probe) with sterile water to void liposomes (COATSOME EL-01-A supplied from NOF Corporation) lyophilized with 8.5% sucrose and mixing homogenously. The FITC-labeled 100-nm polystyrene beads which had not been encapsulated in the liposomes were removed by overlaying the FITC-labeled 100-nm polystyrene beads-encapsulated liposome solution on a separation solution in which density gradient had been made using 3.5 mL of 6% sucrose solution, 3.5 mL of 10% sucrose solution and 3.5 mL of 30% sucrose solution in a centrifuge machine equivalent to an ultracentrifuge swing rotor SW41 supplied from Beckman and ultracentrifuging at 24,000 rpm at 4° C. for one hour, to collect FITC-labeled 100-nm polystyrene beads-encapsulated liposomes. Lyophilized HBsAg particles were obtained by lyophilizing a solution of HBsAg particles in PBS (phosphate buffered saline) containing sucrose at a final concentration of 5% overnight (Vaccine. Apr. 30, 2001;19(23-24):3154-63. Physicochemical and immunological characterization of hepatitis B virus envelope particles exclusively consisting of the entire L (pre-S1+pre-S2+S) protein. Yamada T, Iwabuki H, Kanno T, Tanaka H, Kawai T, Fukuda H, Kondo A, Seno M, Tanizawa K, Kuroda S.). The resulting lyophilized HBsAg particles were fused to the FITC-labeled 100-nm polystyrene beads-encapsulated liposomes by mixing the HBsAg particles with the FITC-labeled 100-nm polystyrene beads-encapsulated liposomes homogenously. As a result, FITC-labeled 100-nm polystyrene beads-encapsulated HBsAg fusion particles were obtained. The fusion of the liposomes to the HBsAg particles was facilitated by heating at 37° C. for one hour after mixing. Only the HBsAg fusion particles could be collected by overlaying this sample on the separation solution in which density gradient had been made using 3.5 mL of 10% sucrose solution, 3.5 mL of 30% sucrose solution and 3.5 mL of 50% sucrose solution and ultracentrifuging at 24,000 rpm at 4° C. for 2 hour in the same way as the above. FIG. 1 is a graph showing separation profile of fractions collected from an upper part of a centrifuge tube after the ultracentrifugation. A peak in the fraction 8 corresponded to the HBsAg fusion particle, and the HBsAg composite particles were obtained by collecting this fraction. Most of BNC constituted the fusion of liposome, and free BNC did nearly not existed. BNC fused liposome was observed under TEM (FIG. 1B). Electron micrograph of BNC fused liposome was shown that BNC surrounded the liposome containing FITC-beads. Average size of BNC was about 200 nm (Table. 1).

TABLE 1

| | Average size |
|---|---|
| Material | (nm) |
| FITC-labeled 100-nm polystyrene beads | 118 |
| FITC-labeled 100-nm polystyrene beads-encapsulated | 123 |
| liposome | |
| BNC fused to the beads-encapsulated liposome | 202 |
| GFP plasmid | 308 |
| GFP plasmid-encapsulated liposome | 154 |
| BNC fused to GFP plasmid-encapsulated liposome | 150 |

As shown in Table 1, the composite particle of the present invention can make the nucleic acid (DNA/RNA) such as GFP plasmid compact (reduce the size) and is suitable for the drug delivery system (DDS).

### Example 2

### Substance Delivery into Hepatic Cancer Cell HepG2 by HBsAg Particle in Which Substance has been Encapsulated Via Liposome

The human hepatic cancer cell HepG2 at an exponential growth phase was seeded in a 96-well plastic plate at 1×10⁴ cells/well, and cultured using MEM (modified Eagle medium) containing 10% fetal bovine serum at 37° C. in the presence of 5% CO₂ overnight. On a subsequent day, the FITC-labeled 100-nm polystyrene beads-encapsulated HBsAg particle was prepared using the FITC-labeled 100-nm polystyrene beads (diameter: 100 nm), the lyophilized liposome and the lyophilized HBsAg particle in the same way as in Example 1. Then, this was added to the above culture of HepG2, which was then cultured at 37° C. in the presence of CO₂ overnight.

The amount of the FITC-labeled 100-nm polystyrene beads introduced into HepG2 was quantified by measuring using a plate reader. Appearances of the introduction of the FITC-labeled 100-nm polystyrene beads in HepG2 were also observed by a confocal laser fluorescent microscope.

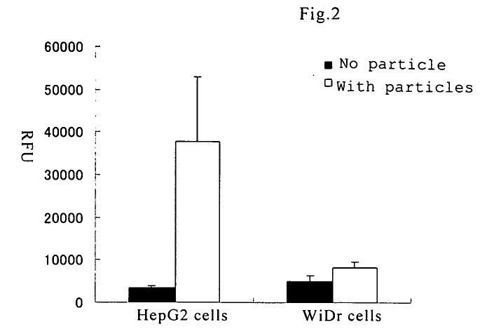

The results of quantifying the FITC-labeled 100-nm polystyrene beads were shown in FIG. 2. Fluorescent photographs of HepG2 were shown in FIG. 3. The results obtained using human large intestine cancer cell WiDr were shown together as the control of HepG2 in FIGS. 2 and 3. From the graph in FIG. 2, in the case of HepG2, the introduction efficiency using the HBsAg particle fused to the liposome was much higher than that using the FITC-labeled 100-nm polystyrene beads-encapsulated liposome alone, and the fluorescent intensity was about 10 times higher. On the contrary, in the case of WiDr, the introduction efficiency was scarcely different between the use of the liposome alone and the use of the HBsAg particle fused to the liposome. Thus, it was found that the specificity of the HBsAg particle for the hepatic cell was retained after the fusion to the liposome. In FIG. 3, the photographs using RITC-labeled 100-nm polystyrene beads in place of the FITC-labeled 100-nm polystyrene beads were shown. In FIG. 3, the introduction of the HBsAg particle fused to the liposome was observed only in HepG2.

From the above, it has been demonstrated that it is possible to deliver the substance with extremely high specificity and efficiency into the human hepatic cell using the HBsAg particle in which the substance has been encapsulated via the liposome of the present invention at a cultured cell level.

### Example 3

### Substance Delivery into Nude Mice Bearing Human Hepatic Cancer by HBsAg Particle in Which the Substance has been Encapsulated Via Liposome

Cancer-bearing mice (Strain: BALB/c, nu/nu, microbiological quality: SPF, sex: male, 5 weeks of age) were obtained by injecting the human hepatic cancer-derived cell NuE-at 2×10⁵ cells subcutaneously at bilateral dorsal portions in nude mice and growing for 2 to 3 weeks until the transplanted cells became a solid cancer with a diameter of 2 cm.

The FITC-labeled 100-nm polystyrene beads-encapsulated HBsAg fusion particle (100 μg) (dissolved in 100 μL of PBS) obtained by the method described in Example 1 was administered in a murine tail vein using a 26 G injection needle. Sixteen hours after the administration, the mouse was anesthetized and perfusion fixation was given thereto according to the standard method. Subsequently, the cancer, liver and kidney were removed, and tissues thereof were fixed and embedded using a resin embedding kit (Technovit 8100).

Specifically, after abdominal section, the left ventricle was stung with a 21 G winged injection needle, and right auricle of the heart was cut and PBS was run to exsanguinate. Subsequently, 4% neutral formaldehyde solution previously cooled on ice was run to fill the tissue with the formaldehyde solution. After removing the tissue, the tissue was immersed in and fixed with the 4% neutral formaldehyde solution at 4° C. for 2 hours, and immersed in 6.8% sucrose-PBS solution at 4° C. overnight. On the subsequent day, the tissue was dehydrated with 100% acetone, then immersed in Technovit 8100 at 4° C. within 24 hours, and left stand at 4° C. after removing from it to perform a polymerization reaction.

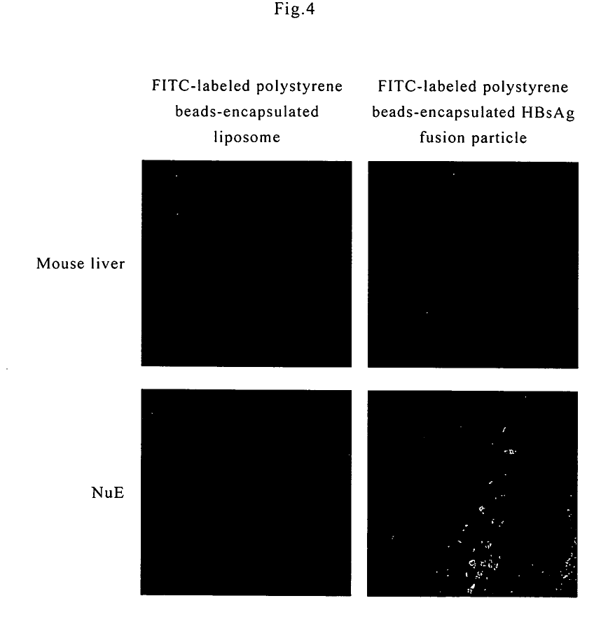

Histological slices were made according to the standard methods, and the fluorescence by the FITC-labeled 100-nm polystyrene beads was compared between the HBsAg particle administration group and the non-administration group by the confocal laser fluorescent microscopy (FIG. 4).

In FIG. 4, the fluorescence derived from the FITC-labeled 100-nm polystyrene beads was observed in the cancer derived from the human hepatic cancer cell NuE in the cancer-bearing mouse. However, no fluorescence was observed in the liver and the kidney simultaneously removed from the same mouse. No fluorescence was observed in the tissues including the cancer in the cancer-bearing mice to which the FITC-labeled 100-nm polystyrene beads alone or the FITC-labeled 100-nm polystyrene beads-encapsulated liposome alone had been administered.

From the above, it has been found that the HBSAg particle in which the substance has been encapsulated via the liposome enables to deliver the substance with extremely high specificity and efficiency to the human hepatic cancer cell at an experimental animal level.

### Example 4

### Substance Delivery into Human Squamous Cell Carcinoma Cell A431 by ZZ tag-Surface Presenting HBSAg Particle (ZZ-HBsAg Particle) in Which the Substance has been Encapsulated Via Liposome

The human squamous cell carcinoma cell A431 at an exponential growth phase was seeded in a 96-well plastic plate at 1×10⁴ cells/well, and cultured using MEM (modified Eagle medium) containing 10% fetal bovine serum at 37° C. in the presence of 5% CO₂ overnight. On the subsequent day, the FITC-labeled 100-nm polystyrene beads-encapsulated ZZ-HBsAg fusion particle was prepared using the FITC-labeled 100-nm polystyrene beads, the lyophilized liposome and the lyophilized ZZ-HBsAg particle (PCT/JP03/03694) in the same way as in Example 1. Subsequently, 8 μg of a monoclonal antibody (anti-hEGFR antibody) against human Epidermal Growth Factor Receptor; hEGFR and 100 μg of the prepared ZZ-HBsAg fusion particle were mixed homogenously and a binding reaction was performed at 4° C. for one hour. The anti-hEGFR antibody presenting ZZ-HBsAg fusion particle obtained by this binding reaction was added to the culture of A431, which was then cultured at 37° C. in the presence of 5% CO₂ overnight.

The amount of the FITC-labeled 100-nm polystyrene beads introduced into A431 was quantified by measuring using the plate reader. Appearances of the introduction of the FITC-labeled 100-nm polystyrene beads were also observed by the confocal laser fluorescent microscope.

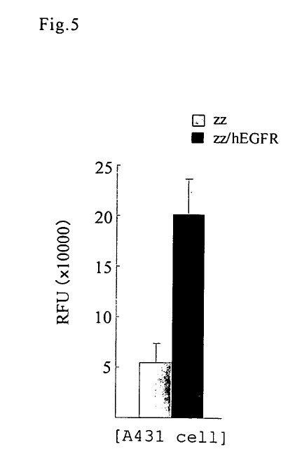

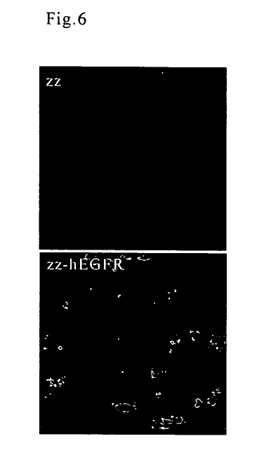

The results of quantifying the FITC-labeled 100-nm polystyrene beads were shown in the graph in FIG. 5. Fluorescent photographs of A431 were shown in FIG. 6. The results obtained from the ZZ-HBsAg fusion particle to which the anti-hEGFR antibody had not been bound were shown together as the control. From the graph in FIG. 5 and the photographs in FIG. 6, it was found that the ZZ-HBsAg fusion particle could deliver the encapsulated FITC-labeled 100-nm polystyrene beads into A431 via the anti-hEGFR antibody. From this result, it was shown that the ZZ-HBsAg particle retained an antibody binding ability of the ZZ tag after being fused to the liposome and could deliver the substance via the antibody bound to the ZZ tag.

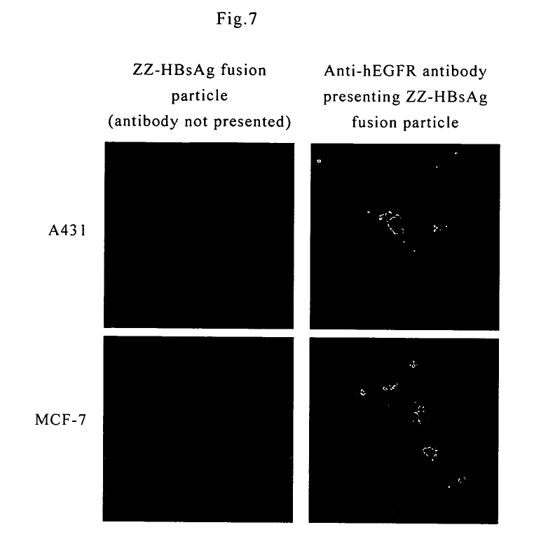

In FIG. 7, the photographs were shown when similarly to the above, the substance was delivered into the breast cancer derived cell MCF-7 which expressed the EGF receptor on the cell surface as with A431 using the RITC-labeled 100-nm polystyrene beads in place of the FITC-labeled 100-nm polystyrene beads. From the results in FIG. 7, it was shown that even the HBsAg particle which presented the foreign functional protein on its surface could encapsulate the substance inside thereof with retaining the function of the particle by using the technique in Example 1.

### Example 5

### Gene Transfection into Human Hepatic Cancer Cell HepG2 by HBsAg Particle in Which the Gene has been Encapsulated Via Liposome

The human hepatic cancer cell HepG2 at an exponential growth phase was seeded in the 96-well plastic plate at 1×10⁴ cells/well, and cultured using MEM (modified Eagle medium) containing 10% fetal bovine serum at 37° C. in the presence of 5% CO₂ overnight. On the subsequent day, a GFP expression plasmid-encapsulated liposome solution was prepared by adding 1.5 mL of a Green Fluorescence Protein; GFP expression plasmid (pEGFP-C1; Clontec) solution prepared at 50 μL/mL with sterile water to the void liposome (COATSOME EL-01-A supplied from NOF Corporation) lyophilized with 8.5% sucrose, mixing homogenously and leaving stand at room temperature for 5 minutes. The HBsAg particle was fused to the GFP expression plasmid-encapsulated liposome by homogenously mixing 150 μL of the prepared GFP expression plasmid-encapsulated liposome solution with 200 μg of the lyophilized HBsAg particle obtained by lyophilizing the HBsAg particle in sucrose at a final concentration of 5% overnight and leaving stand at room temperature for 5 minutes. As a result, the GFP expression plasmid-encapsulated HBsAg fusion particle was obtained. Subsequently, this was added to the culture of HepG2, which was then cultured 37° C. in the presence of 5% CO₂ for 48 hours. After 48 hours, appearances of GFP expressed in the cell by the transfected GFP expression plasmid were observed by the confocal laser fluorescent microscope.

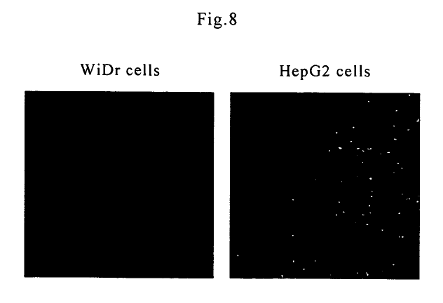

The fluorescent photographs of HepG2 were shown in FIG. 8. The result obtained from the human large intestine cancer cell WiDr was also shown as the control in FIG. 8. From the results in FIG. 8, the gene transfection by the HBsAg fused to the liposome was observed only in HepG2, and no fluorescent was observed in WiDr.

From the above, it has been shown that in accordance with the present invention, the gene can be transfected with extremely high specificity and efficiency into the human hepatic cell using the HBsAg particle in which the gene has been encapsulated via liposome at the cultured cell level.

### Example 6

Ex vivo and in vivo Delivery of 100-nm Polystyrene Beads with BNC Fused Liposome

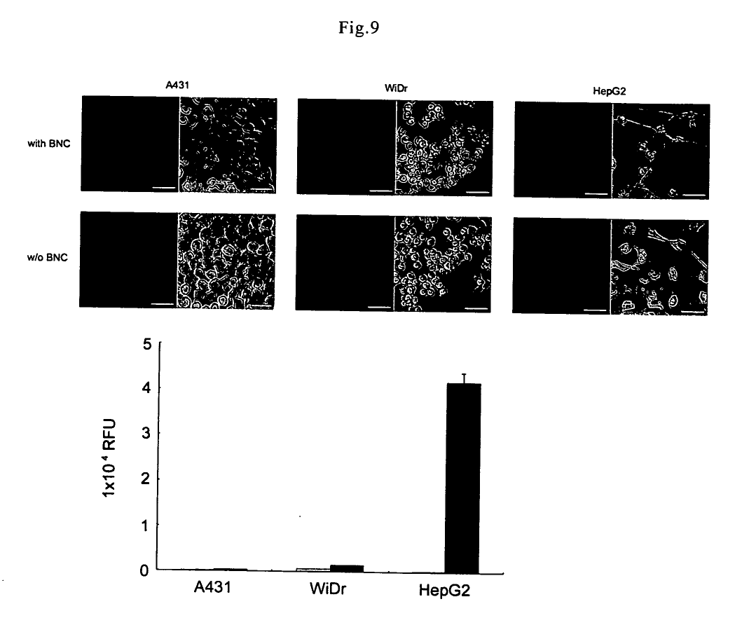

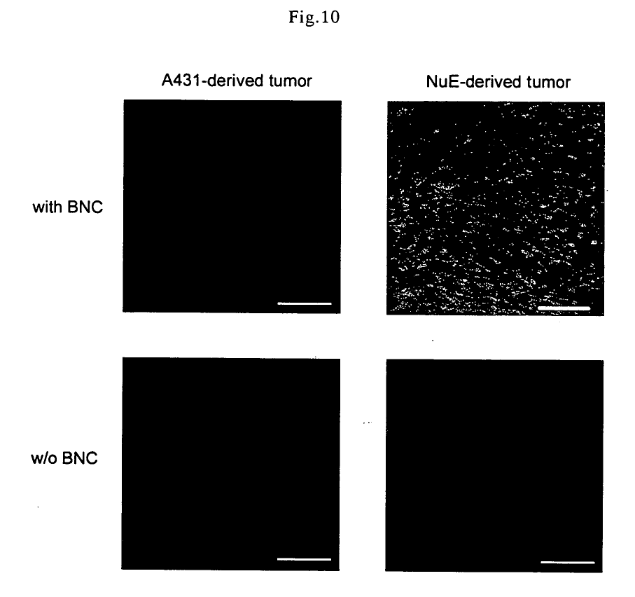

BNC fused liposome containing rhodamine-labeled 100-nm polystyrene beads (Rho-beads) was used without separation by ultracentrifugation, because BNC was able to fuse with liposome almostly. 10 μg of BNC fused liposome containing 1 μg of Rho-beads were used to transfect into about 5×10⁴ cells of HepG2 cells, WiDr cells, and A431 cells. After 6 h, fluorescence was observed specifically in HepG2 cell, not in WiDr and A431 cells (FIG. 9). In addition, these BNC were injected into xenograft model bearing hepatic NuE cells and A431 cells. 100 μg of BNC fused liposome containing 25 μg of Rho-beads per mouse were used. After 16 h, fluorescence was observed in NuE-derived tumor, but A431-derived tumor. To confirm the exist site of Rho-beads, FITC-labeled tomato lectin was injected before scarify. Rho-beads existed around the blood vessels shown as a green color in FIG. 10.

### Example 7

Ex vivo and in vivo Gene Delivery with BNC Fused Liposome

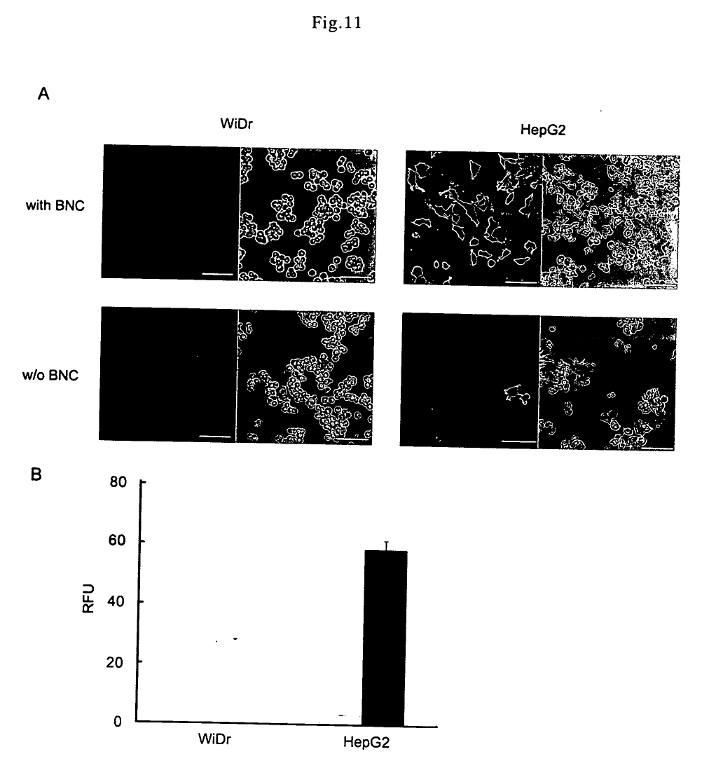

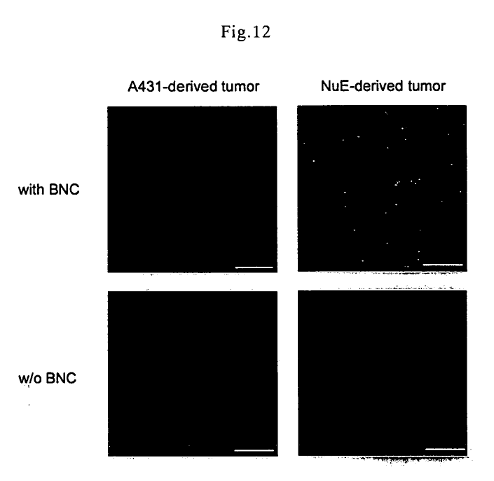

To incorporate DNA into BNC, DNA was first encapsulated in cationic liposome and that was fused with BNC in the same way as the incorporation of beads. BNC fused liposome containing GFP plasmid (pEGFP-C1) was added to HepG2 and WiDr cells (2×10⁵ cells/well). 2 μg of GFP plasmid was incorporated into 10 μg of BNC. On day 2 after transfection, GFP expression was significantly observed to HepG2 cells treated BNC (FIG. 11). In xenograft model, 50 μg of BNC was injected. After 5 days, mouse liver, mouse kidney, NuE-derived tumor, and A431-derived tumor were harvested. Although this amount was reduced compared to the amount of BNC containing beads per mouse, GFP expression was observed in NuE-derived tumor, not A431-derived tumor (FIG. 12). Absolutely, GFP expression was not observed in mouse liver and kidney (data not shown).

### Example 8

Efficient ex vivo Delivery of 35-kbp GFP Plasmid with BNC Fused Liposome

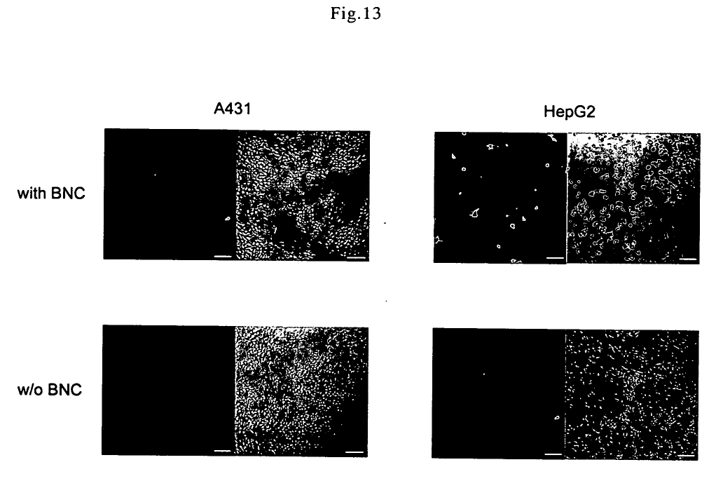

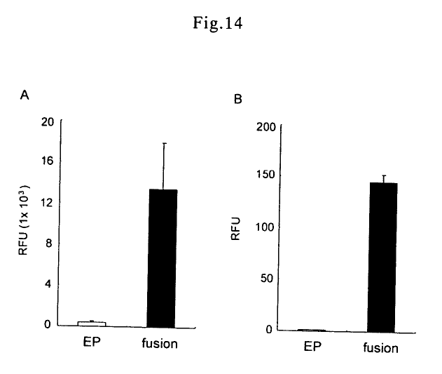

4.7-kbp pEGFP-C1 was efficiently incorporated into BNC and delivered to HepG2 or NuE cells ex vivo or in vivo. Furthermore, 6.4-kbp pcDNA6.2/C-EmGFP was also delivered to hepatocytes (data not shown). To determine size limits of DNA, we used pAD/CMV-GFP (about 35 kbp) for enclosure within BNC. HepG2 cells and A431 cells (5×10⁴ cells/well) were seeded and BNC fused liposome containing 2 μg of DNA was transferred to HepG2 and A431 cells next day. After 48 h, GFP expression was predictably observed in HepG2 cells, not A431 cells (FIG. 13). Efficiency of transfection was about 10% (n=400) although the efficiency of transfection by electroporation was <1% .

What is claimed is:

1. A method of producing a composite particle of a nanoparticle and a liposome encapsulating a substance to be introduced, characterized in that a hollow nanoparticle comprising a hepatitis B virus protein or a modification thereof is fused to the liposome in which the substance to be introduced has been encapsulated.

2. The method according to claim 1 wherein a particle diameter of said hollow nanoparticle is about 80 to about 130 nm.

3. The method according to claim 1 wherein a particle diameter of said composite particle is about 130 to about 500 nm.

4. The method according to claim 1 wherein a particle diameter of said composite particle is about 150 to about 400 nm.

5. The method according to claim 1 wherein the hollow nanoparticle comprising the hepatitis B virus protein or the modification thereof is composed of about 70 to about 90 parts by weight of the hepatitis B virus protein or the modification thereof, about 5 to about 15 parts by weight of lipid and about 5 to about 15 parts by weight of sugar chain.

6. The method according to any one of claims 1 to 5 wherein said hollow nanoparticle has been previously lyophilized or spray-dried.

7. A composite particle comprising a nanoparticle portion comprises a hepatitis B virus protein or a modification thereof, lipid and sugar chain, and an exogenous substance encapsulated in the nanoparticle portion.

8. The composite particle according to claim 7 wherein a particle diameter of said composite particle is about 150 to about 400 nm.

9. The composite particle according to claim 7 or 8, wherein the nanoparticle portion comprising about 70 to about 90 parts by weight of the hepatitis B virus protein or the modification thereof, about 6 to about 75 parts by weight of the lipid and 5 to 15 parts by weight of the sugar chain.

10. The composite particle according to claim 9, wherein said lipid comprises about 5 to about 15 parts by weight of lipid which is the component of a membrane of an eukaryotic cell and about 1 to about 60 parts by weight of lipid which is the component of a liposome.

11. The composite particle according to claim 9, wherein the nanoparticle portion comprises about 70 to about 90 parts by weight of the hepatitis B virus protein or the modification thereof, about 5 to about 15 parts by weight of lipid which is the component of a membrane of an eukaryotic cell, about 2 to about 30 parts by weight of lipid which is the component of a liposome and about 5 to about 15 parts by weight of the sugar chain.

12. A composite particle of a nanoparticle and a liposome encapsulating a substance to be introduced, obtainable by the method according to any one of claims 1 to 5.

13. A method of introducing a substance to be introduced into a target cell, including allowing the composite particle according to any of claims 7 to 11 or the composite particle according to claim 12 to act upon the target cell.
